(19) United States
(12) Patent Application Publication
(10) Pub. No.: US 2023/0310730 A1
(43) Pub. Date: Oct. 5, 2023

(54) SUCTION INSTRUMENT AND METHOD FOR VIDEO-ASSISTED THORACOSCOPIC SURGERY

(71) Applicants: Mayo Foundation for Medical Education and Research, Rochester, MN (US); Scanlan International, Inc., St. Paul, MN (US)

(72) Inventors: Shanda H. Blackmon, Rochester, MN (US); Jean E. Daavettila, St. Paul, MN (US); Robert E. Kohler, Lake Elmo, MN (US)

(21) Appl. No.: 18/129,159

(22) Filed: Mar. 31, 2023

Related U.S. Application Data
(60) Provisional application No. 63/325,710, filed on Mar. 31, 2022.

(51) Int. Cl. A61M 1/00; A61M 13/00

(52) U.S. Cl. CPC A61M 1/85; A61M 13/003; A61M 2210/1039; A61B 2217/005; A61M 25/007

(57) ABSTRACT

Instruments and methods for removing tissue from a patient's thoracic cavity during surgery, for example VATS, while reducing re-inflation of the lungs. Embodiments include an instrument comprising a first structure defining a first lumen and a second structure defining a second lumen. The first structure includes a first proximal end portion comprising a first proximal port to the first lumen, and a first distal end portion comprising a first distal port to the first lumen. The second structure includes a second proximal end portion comprising a second proximal port to the second lumen, and a second distal end portion comprising a second distal port to the second lumen. The instrument is configured to be inserted into a patient with the first and second distal end portions located in the cavity to facilitate suction of tissue through the first lumen, and entry of make-up gas into the cavity through the second lumen.

### FIELD

This disclosure relates generally to aspiration or suction instruments and methods for use in connection with surgical procedures, such as for example video-assisted thoracoscopic surgery (VATS).

### BACKGROUND

The use of instruments to produce negative pressure or suction to aspirate or remove fluid and other tissue such as blood during surgical procedures is generally known. For example, video-assisted thoracoscopic surgery (VATS) is a minimally invasive surgical technique that may be used to diagnose and treat a variety of conditions involving the patient's thorax or chest area. During VATS, a video camera such as a thoracoscope and other surgical instruments are inserted into the patient's chest through one or more small incisions in the chest wall. The thoracoscope produces images of the surgical field that are displayed on a video monitor and used by the surgeon performing the procedure to guide the surgery During VATS procedures the patient's lungs may be deflated to increase the available space within the thoracic cavity for performing the procedure.

Suction or aspiration instruments may be placed into the thoracic cavity during VATS to remove tissue such as blood during the procedure. However, the negative pressure created in the thoracic cavity by the suction instruments may cause the patient's lungs to re-inflate.

There remains a continuing need for improved surgical instruments and associated methods and procedures. For example, there is a need for improved suction instruments and associated methods for removing blood and other tissues from surgical sites. Such instruments and methods that can be used during VATS would be desirable. In particular, instruments and methods that can effectively remove blood or other tissue during VATS without causing lung re-inflation would be especially advantageous.

### SUMMARY

Improved surgical suction instruments and methods are described herein. The surgical instruments and methods effectively remove blood and other tissue from surgical fields. The instruments and methods are particularly well suited for video-assisted thoracoscopic surgery (VATS), including such procedures that are performed with the patient's lungs partially or wholly deflated. The instruments and methods may, for example effectively remove blood and other tissue from a patient's thoracic cavity during VATS, while minimizing or otherwise reducing re-inflation of the patent's lungs.

A first example is an instrument, comprising: a first structure defining a first lumen, the first structure including: a first proximal end portion comprising a first proximal port to the first lumen, wherein the first proximal port is configured to be coupled to a suction source; a first distal end portion comprising a first distal port to the first lumen, wherein the first distal end portion including the first distal port are configured to be located at a site within a cavity of a subject; and wherein the first structure causes tissue and/or gas to be drawn into the first distal port and through the first lumen when the first proximal end portion is coupled to the suction source; and a second structure defining a second lumen, the second structure including: a second proximal end portion comprising a second proximal port to the second lumen, wherein the second proximal end portion including the second proximal port are configured to be located outside the cavity of the subject when the first distal end portion including the first distal port of the first structure are located within the cavity of the subject; and a second distal end portion comprising a second distal port to the second lumen, wherein the second distal end portion including the second distal port are configured to be located within the cavity of the subject when the first distal end portion including the first distal port of the first structure are located within the cavity of the patient; and wherein the second structure allows make-up gas to flow into the second proximal port, through the second lumen and out the second distal port, and into the cavity of the subject, when tissue and/or gas is drawn into the first distal port of the first lumen of the first structure.

In some embodiments of the first example, the first structure defines a first length between the first proximal end portion and the first distal end portion; and the second structure defines a second length between the second proximal end portion and the second distal end portion. In some embodiments, the first and second structures are coupled to one another along the first and second lengths.

Any or all embodiments of the first example may further comprise a handle on the first proximal end portion of the first structure. In some embodiments, the first length is greater than the second length. In some embodiments, the handle is located on the first structure proximal of the second proximal end portion of the second structure.

In any or all embodiments of the first example, the first proximal port is located adjacent to or proximal to a distal end of the handle.

In any or all embodiments of the first example, the first length is greater than the second length.

In any or all embodiments of the first example, the first distal port of the first structure includes one or more openings; the second distal port of the second structure includes one or more openings; and the second proximal port of the second structure includes one or more openings. In some embodiments, at least one of the one or more openings of the second distal port is located proximal to a proximal-most opening of the one or more openings of the first distal port. In some embodiments, each of the at least one or more openings of the second distal port is located proximal to a proximal-most opening of the one or more openings of the first distal port.

In some embodiments of the first example, the second distal port of the second structure includes a plurality of openings spaced apart from one another along a length of the second structure, including one or more openings through a side of the second structure.

In some embodiments of the first example, the second distal port of the second structure includes an opening on a distal end face of the second structure.

In some embodiments of the first example, the second proximal port of the second structure includes one or more openings. In some embodiments, the second proximal port of the second structure includes a plurality of openings spaced apart from one another along a length of the second structure, including one or more openings through a side of the second structure. In some embodiments, the second proximal port of the second structure includes an opening on a proximal end face of the second structure.

In some embodiments of the first example, the first distal port of the first structure includes one or more openings through a side of the first structure.

In some embodiments of the first example, the first distal port of the first structure includes an opening on a distal end face of the first structure.

In any or all embodiments of the first example, the second structure is configured to allow make-up gas to be drawn into the second proximal port, through the second lumen and out the second distal port, and into the cavity of the subject, in response to tissue and/or gas being drawn into the first distal port of the first lumen of the first structure.

In any or all embodiments of the first example, the second proximal port of the second structure is configured to be exposed to atmospheric pressure outside the cavity of the subject.

In any or all embodiments of the first example, the second proximal port of the second structure is configured to be coupled to a source of gas at a pressure greater than atmospheric pressure.

A second example is an instrument, comprising: a first structure defining a first lumen, including: a first proximal end portion comprising a first proximal port to the first lumen; and a first distal end portion comprising a first distal port to the first lumen; and a second structure defining a second lumen, including: a second proximal end portion comprising a second proximal port to the second lumen; and a second distal end portion comprising a second distal port to the second lumen; and wherein the instrument is configured to be inserted into a subject with the first and second distal end portions located in a cavity of the subject to facilitate suction of tissue and/or gas from the cavity through the first lumen and entry of make-up gas into the cavity through the second lumen.

A third example is a method for performing a surgical procedure on a subject, comprising: inserting an instrument on accordance with any of the embodiments of the first or second examples into a cavity of the subject, optionally a cavity of the subject's thorax; positioning the instrument so the distal end portion of the first structure and the distal end portion of the second structure are within the cavity, and the proximal end portion of the first structure and the proximal end portion of the second structure are outside of the cavity; applying a source of suction to the first proximal port of the first structure to cause tissue to be withdrawn from the cavity through the first lumen of the first structure; and causing make-up gas to flow into the cavity through the second lumen of the second structure while applying the source of suction to the first structure.

In embodiments of the third example, positioning the instrument includes positioning the instrument so the second proximal port of the second structure is exposed to ambient atmosphere; and causing make-up gas to flow into the cavity includes casing the make-up gas to be drawn into the cavity through the second lumen in response to the withdrawal of the tissue through the first lumen.

In any or all embodiments of the third example, the method is performed in a cavity of the subject including a deflated lung of the subject. Some embodiments further include operating the instrument in a manner to not cause re-inflation of the lung of the subject.

Some embodiments of the third example further comprise applying a source of gas at a pressure greater than atmospheric pressure to the second proximal port of the second structure.

A fourth example is a method, comprising: providing a first structure defining a first lumen and including a first proximal end portion including a first proximal port to the first lumen and a first distal end portion including a first distal port to the first lumen; providing a second structure defining a second lumen and including a second proximal end portion including a second proximal port to the second lumen and a second distal end portion including a second distal port to the second lumen; inserting the first structure into a cavity in a subject through an opening in the subject's body, including positioning the first distal port in the cavity; applying suction to the first proximal port of the first structure to suction tissue from the cavity through the first distal port and the first lumen; inserting the second structure into the cavity of the subject through an opening in the subject's body, including positioning the second distal port in the cavity, and positioning the second proximal port outside of the cavity; and wherein make-up gas flows into the cavity through the second lumen and the second distal port while the suction is applied to the first proximal port of the first structure.

In embodiments of the fourth example, inserting the first and second structures into the cavity comprises inserting the first and second structures into the cavity through the same opening in the subject's body.

In any or all embodiments of the fourth example, inserting the first and second structures into the cavity comprises simultaneously inserting the first and second structures into the cavity.

In any or all embodiments of the fourth example, providing the first structure and providing the second structure comprises providing a unitary member including both the first structure and the second structure.

In any or all embodiments of the fourth example, inserting the second structure into the cavity includes causing the second proximal port to be exposed to ambient atmosphere.

In any or all embodiments of the fourth example, inserting the second structure into the cavity includes causing the second distal port to be located at a position that is spaced-apart from the first distal port.

Any or all embodiments of the fourth example may further include facilitating the flow of make-up gas into the cavity through the second lumen of the second structure in response to the suction of tissue through the first lumen of the first structure.

Any or all embodiments of the fourth example may further include applying a source of gas at a pressure greater than ambient pressure to the second proximal port of the second structure.

### BRIEF DESCRIPTION OF THE DRAWINGS

FIG. 1 is a side view of a vented suction instrument, in accordance with embodiments.

FIG. 2 is an isometric view of the instrument shown in FIG. 1, showing top and side portions of the instrument, in accordance with embodiments.

FIG. 3 is a distal end view of the instrument shown in FIG. 1, in accordance with embodiments.

FIGS. 4A-4C diagrammatically illustrate a surgical site of a patient and a method by which the instrument shown in FIG. 1 may be used in connection with the surgical site.

### DETAILED DESCRIPTION

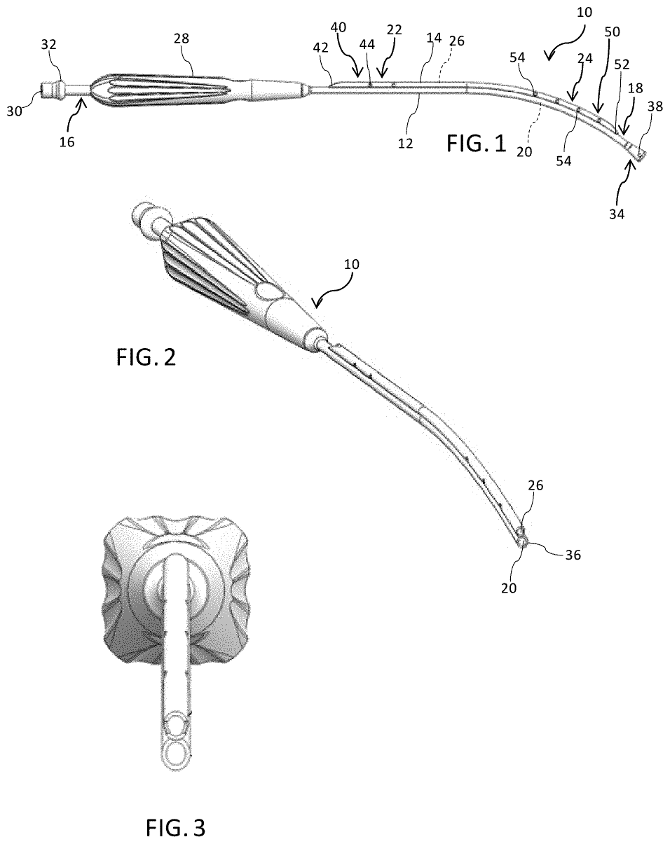

FIGS. 1-3 illustrate a vented suction instrument 10 in accordance with embodiments. As described in greater detail below, instrument 10 may be used in connection with surgical procedures to suction, aspirate or otherwise remove tissues such as blood and/or other fluids from the surgical site. The instrument 10 is vented to the ambient atmosphere or otherwise coupled to a source of air or other gas to provide make-up gas to the surgical site as the tissue is suctioned. Enhanced tissue removal from the surgical site can thereby be achieved, improving efficacy of the surgical procedures. In embodiments, the instrument 10 is used in connection with minimally-invasive surgical procedures, and may be inserted into a cavity of within the patient through an opening in a wall of the patient's body, and positioned at the surgical site within the cavity. For example, the instrument 10 may be used in connection with video-assisted thoracoscopic surgery (VATS) during procedures within the thorax or chest of the patent. During VATS procedures the lungs of the patient may be partially or fully deflated to increase the size of the cavity surrounding the surgical site. The vented suction instrument 10 is particularly advantageous for use with such VATS procedures because the make-up air provided by the instrument may reduce undesired re-inflation of the lungs.

As shown in FIGS. 1-3, the illustrated embodiments of the instrument 10 include a first tubular structure 12 and a second tubular structure 14. The first tubular structure 12 has a proximal end portion 16 and a distal end portion 18, and defines a first lumen 20 that extends between the proximal and distal end portions 16, 18. The second tubular structure 14 has a proximal end portion 22 and a distal end portion 24, and defines a second lumen 26 that extends between the proximal and distal end portions 22, 24. In the illustrated embodiments the first tubular structure 12 and second tubular structure 14 are portions of a unitary structure. A handle 28 may be located at a proximal end portion of the instrument 10, and is shown on the proximal end portion 16 of the first tubular structure 12 in the illustrated embodiments. The first and second tubular structures 12 and 14 and the handle 28 may be manufactured from any material suitable for the application of the instrument 10, such as for example polymer materials and/or metals. The instrument 10 may be disposable, or suitable for sterilization and reuse.

In embodiments, for example, the tubular members may be initially manufactured as separate structures, and then joined together at one or more locations along their lengths to form the unitary structure. In other embodiments (not shown), the first and second tubular structures 12 and 14 may be movable with respect to one another (e.g., the second tubular structure may be configured to slide to one or more different positions along the length of the first tubular structure). Although the second tubular structure 14 is shown on one side (e.g., a top side) of the first tubular structure 12 in the illustrated embodiments, in other embodiments the second tubular structure is located on one or more other sides (e.g., a bottom side) of the first tubular structure. Although shown generally parallel to one another in the illustrated embodiments, in other embodiments the first and second tubular structures 12 and 14 may be oriented or extend in other configurations with respect to one another.

A proximal port 30 on the proximal end portion 16 of the first tubular structure 12 defines one or more openings in communication with the first lumen 20, and is located proximal of the handle 28 in the illustrated embodiments. A connector 32 may be included on the proximal port 30 to facilitate coupling of the first tubular structure 12 to a suction source or other relatively negative pressure source. The proximal port 30 defines a single opening into the first lumen 20 in embodiments of the instrument 10 such as those shown in FIGS. 1-3.

A distal port 34 on the distal end portion 18 of the first tubular structure 12 defines one or more openings in communication with the first lumen 20. In the illustrated embodiments the distal port 34 defines an opening 36 on the distal end face of the first tubular structure 12, and one or more openings 38 on the side wall of the first tubular structure (one opening 38 is shown in FIG. 1). In other embodiments (not shown) the distal port 34 defines one or more openings such as 36 on only the distal end face of the first tubular structure 12, or one or more openings such as 38 on only the side wall of the first tubular structure. Embodiments may have no openings such as 38 on the side wall of the first tubular structure 12.

A proximal port 40 on the proximal end portion 22 of the second tubular structure 14 defines one or more openings in communication with the second lumen 26. In the illustrated embodiments the proximal port 40 defines an opening 42 on the proximal end face of the second tubular structure 14, and one or more openings 44 on the side wall of the second tubular structure. A plurality of the side wall openings 44 are shown in the illustrated embodiments, and the openings 44 are spaced apart from one another and the end opening 42 along the length of the second tubular structure 14. In other embodiments the proximal port 40 on the proximal end portion 22 of the second tubular structure 14 includes other configurations of openings such as 42 and/or 44. For example, in embodiments (not shown), the proximal port 40 defines one or more openings such as 42 on only the proximal end face of the second tubular structure 14, or one or more openings such as 44 on only the side wall of the second tubular structure. Embodiments may have no openings such as 44 on the side wall of the second tubular structure 14.

A distal port 50 on the distal end portion 24 of the second tubular structure 14 defines one or more openings in communication with the second lumen 26. In the illustrated embodiments the distal port 50 defines an opening 52 on the distal end face of the second tubular structure 14, and one or more openings 54 on the side wall of the second tubular structure. A plurality of the side wall openings 54 are shown in the illustrated embodiments, and the openings 54 are spaced apart from one another and the end opening 52 along the length of the second tubular structure 14. In other embodiments the distal port 50 on the distal end portion 24 of the second tubular structure 14 includes other configurations of openings such as 52 and/or 54. For example, in embodiments (not shown), the distal port 50 defines one or more openings such as 52 on only the distal end face of the second tubular structure 14, or one or more openings such as 54 on only the side wall of the second tubular structure. Embodiments may have no openings such as 54 on the side wall of the second tubular structure 14.

In the illustrated embodiments, the second tubular structure 14 has a length that is shorter than a length of the first tubular structure 12. The distal port 50 of the second tubular structure 14 is located proximally of the distal port 34 of the first tubular structure 12 in the illustrated embodiments. By these embodiments all of the openings such as 52 and 54 defined by the distal port 50 of the second tubular structure 14 are located proximal to all of the openings such as 36 and 38 defined by the distal port 34 of the first tubular structure 12. Other embodiments (not shown) may have other positional relationships of the distal ports 34 and 50 and the openings defined by those ports. For example, the distal port 50 of the second tubular structure 14 may be located adjacent to or distal to the distal port 34 of the first tubular structure 12. As described below, in embodiments the proximal port 40 of the second tubular member 14 is located sufficiently proximal to the distal port 34 of the first tubular member 12 that during use of the instrument 10, the proximal port 40 is positioned at atmospheric pressure, such as for example at locations outside of the body of the patient on which the instrument is being used. In other embodiments (not shown), the proximal port 40 is configured to be coupled to a source of pressurized gas.

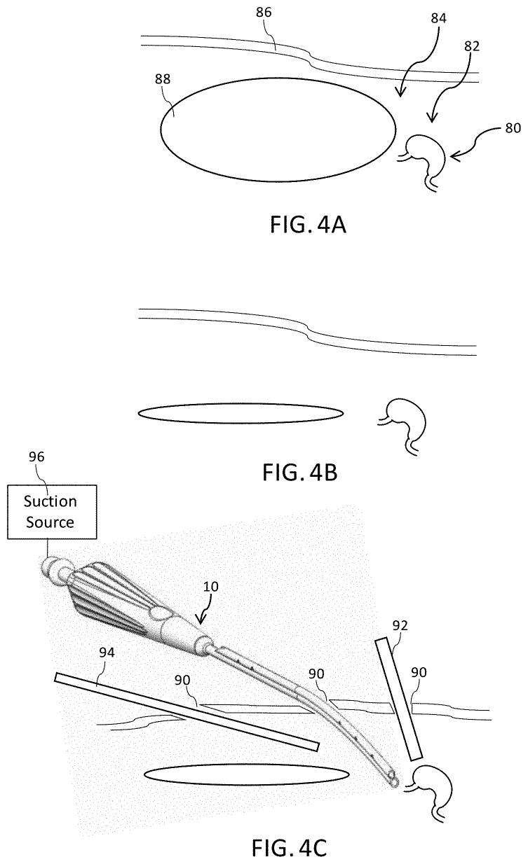

FIGS. 4A-4C are diagrammatic illustrations that can be used to describe a surgical procedure using the instrument 10 in accordance with embodiments. A minimally-invasive VATS procedure is described for purposes of example. Use of instrument 10 with other minimally-invasive or other procedures is contemplated. As shown, the surgical procedure is performed at a surgical site 80 within a cavity 82 in the thorax or chest 84 of the patient or other subject. The cavity 82, which may be defined in part by one or more walls such as 86 of the patient's body, may include a lung 88 of the patient. FIG. 4A diagrammatically illustrates the lung 88 of the patient in an inflated state. In connection with the VATS procedure, the lung 88 of the patient may be deflated, as shown diagrammatically in FIG. 4B, to increase the amount of space in the cavity 82 around the surgical site 80. As shown in FIG. 4C, during the surgical procedure one or more openings 90 through wall 86 and into the cavity 82 are made to provide access to the surgical site 80. Although the illustrated embodiments show three openings 90, other embodiments of the surgical procedure may include more or fewer such openings.

The vented suction instrument 10 and typically one or more other instruments or devices such as those shown diagrammatically at 92 and 94 are inserted into the cavity 82 through the openings 90. Nonlimiting examples of instruments 92 and 94 includes thoraco scopes, biopsy forceps, chest tube passers, clamps, dissectors, lung graspers, needle holders, scissors, suture pullers and suture cutters. Instruments such as 92 and 94 can be inserted, manipulated, positioned and/or used during the surgical procedure, for example at the surgical site 80, in conventional or otherwise known manners.

As described above, the instrument 10 is inserted into the cavity 82 through an opening 90. The first and second tubular structures 12 and 14 are inserted into the cavity 82 by this step, and the instrument 10 is manipulated to position the distal end portions 18 and 24 of the instrument at desired locations within the cavity, such as for example adjacent the surgical site 80. When inserting embodiments of the instrument 10 comprising the first and second tubular structures 12 and 14 as integral members, the first and second tubular structures are effectively simultaneously inserted into the cavity 82. In other embodiments (not shown), one of the first and second tubular structures 12 or 14 may be inserted or positioned at least in part separately and/or individually with respect to the other of the first or second tubular structures.

The distal ports 34 and 50 of the first and second tubular members 12 and 14, and preferably all of the openings such as 36, 38, 52 and 54 of the distal ports, will typically be positioned within the cavity 82 by these actions. As shown in FIG. 4C, when the instrument 10 is inserted into the cavity 82 and positioned at an operative position for the surgical procedure, the proximal port 40 of the second tubular structure 14, and preferably all of the openings such as 42 and 44 of the proximal port, are located outside of cavity 82.

When it is desired to operate the instrument 10 to remove fluids such as blood or other tissues and/or gases or unwanted substances from the cavity 82 and/or surgical site 80, a source of relatively negative pressure such as the suction source 96 shown in FIG. 4C is coupled to the proximal port 30 of the first tubular structure 12. The unwanted substances will then be drawn into the first lumen 20 through the distal port 34 of the first tubular structure 12 because of the relatively negative pressure generated by the suction source 96, and removed from the cavity 82 and/or surgical site 80. Air and/or other gas within the cavity 82 may also be removed from the cavity 82 through the first tubular structure 12 along with the unwanted substances.

The removal of gas and/or unwanted substances from the cavity 82 may tend cause relatively negative pressures in the cavity (e.g., with respect to the ambient environment pressure outside of the cavity). The distal port 50 of the second tubular structure 12 may be exposed to any such negative pressures in the cavity 82. Gas, such as air in the ambient environment, to which the proximal port 40 of the second tubular structure 14 is exposed, will be drawn into the second lumen 26 of the second tubular structure through the proximal port 40, and will flow into the cavity 82 from the second lumen though the distal port 50. The flow of gas into the cavity 82 through the second tubular structure 14 may partially or fully make up for the gas and/or tissue removed from the cavity 82 though the first tubular structure 12, and thereby reduces, minimizes or prevents any such negative pressures that might otherwise occur within the cavity 82. Because of the reduction or prevention of relatively negative pressures within the cavity 82, effects of any such negative pressures that might have otherwise tended to reduce the size of the cavity 82 can be reduced or prevented. For example, any tendencies of the lung 88 to reinflate due to negative pressures within the cavity may be reduced or prevented.

In other embodiments, the proximal port 40 of the second tubular structure 14 may be connected to a source of gas (not shown) having a pressure greater than ambient pressure (e.g., a positive pressure). During the use of such embodiments of an instrument 10, the gas source may cause gas to flow into the cavity 82 to cause insufflation and to expand the cavity 82.

The inclusion of multiple openings on the proximal port 22 and distal port 24 of the second tubular structure 14 (e.g., openings 42, 44, 52, 54) may enhance the above-described functionality of the tubular structure. For example, if one or more of the proximal openings 42 or 44 is closed during the operation of the instrument 10, such as for example if the one or more openings is covered by other structures in the surgical field such as a drape or the hand of the surgeon or a nurse, one or more others of the proximal openings may be open to the second lumen 26. If one or more of the distal openings 52 or 54 is closed during the operation of the instrument 10, such as for example if the one or more opening is plugged by tissue or covered due to engagement with structures at the surgical site 80, one or more others of the distal openings may be open to the second lumen 26.

Instruments such as 10 offer important advantages. As described above, for example, they can provide enhanced efficacy to surgical procedures.

It is to be understood that the above description is intended to be illustrative, and not restrictive. Many other embodiments will be apparent to those of skill in the art upon reading and understanding the above description. It is contemplated that features described in association with one embodiment are optionally employed in addition or as an alternative to features described in or associated with another embodiment. The scope of the invention should, therefore, be determined with reference to the appended claims, along with the full scope of equivalents to which such claims are entitled.

1. An instrument, comprising:
a first structure defining a first lumen, the first structure including:
a first proximal end portion comprising a first proximal port to the first lumen, wherein the first proximal port is configured to be coupled to a suction source;
a first distal end portion comprising a first distal port to the first lumen, wherein the first distal end portion including the first distal port are configured to be located at a site within a cavity of a subject; and
wherein the first structure causes tissue and/or gas to be drawn into the first distal port and through the first lumen when the first proximal end portion is coupled to the suction source; and
a second structure defining a second lumen, the second structure including:
a second proximal end portion comprising a second proximal port to the second lumen, wherein the second proximal end portion including the second proximal port are configured to be located outside the cavity of the subject when the first distal end portion including the first distal port of the first structure are located within the cavity of the subject; and
a second distal end portion comprising a second distal port to the second lumen, wherein the second distal end portion including the second distal port are configured to be located within the cavity of the subject when the first distal end portion including the first distal port of the first structure are located within the cavity of the patient; and
wherein the second structure allows make-up gas to flow into the second proximal port, through the second lumen and out the second distal port, and into the cavity of the subject, when tissue and/or gas is drawn into the first distal port of the first lumen of the first structure.

2. The instrument of claim 1, wherein:
the first structure defines a first length between the first proximal end portion and the first distal end portion; and
the second structure defines a second length between the second proximal end portion and the second distal end portion.

3. The instrument of claim 2, wherein the first and second structures are coupled to one another along the first and second lengths.

4. The instrument of claim 3, further comprising a handle on the first proximal end portion of the first structure.

5. The instrument of claim 4, wherein the first length is greater than the second length.

6. The instrument of claim 5, wherein the handle is located on the first structure proximal of the second proximal end portion of the second structure.

7. The instrument of claim 4, wherein the first proximal port is located adjacent to or proximal to a distal end of the handle.

8. The instrument of claim 2, wherein the first length is greater than the second length.

9. The instrument of claim 1, wherein:
the first distal port of the first structure includes one or more openings;
the second distal port of the second structure includes one or more openings; and
the second proximal port of the second structure includes one or more openings.

10. The instrument of claim 9, wherein at least one of the one or more openings of the second distal port is located proximal to a proximal-most opening of the one or more openings of the first distal port.

11. The instrument of claim 10, wherein each of the at least one or more openings of the second distal port is located proximal to a proximal-most opening of the one or more openings of the first distal port.

12. The instrument of claim 9, wherein the second distal port of the second structure includes a plurality of openings spaced apart from one another along a length of the second structure, including one or more openings through a side of the second structure.

13. The instrument of claim 9, wherein the second distal port of the second structure includes an opening on a distal end face of the second structure.

14. The instrument of claim 9, wherein the second proximal port of the second structure includes one or more openings.

15. The instrument of claim 14, wherein the second proximal port of the second structure includes a plurality of openings spaced apart from one another along a lengthy of the second structure, including one or more openings through a side of the second structure.

16. The instrument of claim 14, wherein the second proximal port of the second structure includes an opening on a proximal end face of the second structure.

17. The instrument of claim 9, wherein first distal port of the first structure includes one or more openings through a side of the first structure.

18. The instrument of claim 9, wherein the first distal port of the first structure includes an opening on a distal end face of the first structure.

19. The instrument of claim 1, wherein the second structure is configured to allow make-up gas to be drawn into the second proximal port, through the second lumen and out the second distal port, and into the cavity of the subject, in response to tissue and/or gas being drawn into the first distal port of the first lumen of the first structure.

20. The instrument of claim 1, wherein the second proximal port of the second structure is configured to be exposed to atmospheric pressure outside the cavity of the subject.

21. The instrument of claim 1, wherein the second proximal port of the second structure is configured to be coupled to a source of gas at a pressure greater than atmospheric pressure.

22. An instrument, comprising:
a first structure defining a first lumen, including:
a first proximal end portion comprising a first proximal port to the first lumen; and
a first distal end portion comprising a first distal port to the first lumen; and
a second structure defining a second lumen, including:
a second proximal end portion comprising a second proximal port to the second lumen; and
a second distal end portion comprising a second distal port to the second lumen; and
wherein the instrument is configured to be inserted into a subject with the first and second distal end portions located in cavity of the subject to facilitate suction of tissue and/or gas from the cavity through the first lumen and entry of make-up gas into the cavity through the second lumen.

23. A method for performing a surgical procedure on a subject, comprising:
inserting the instrument of claim 1 into a cavity of the subject, optionally a cavity of the subject's thorax;
positioning the instrument so the distal end portion of the first structure and the distal end portion of the second structure are within the cavity, and the proximal end portion of the first structure and the proximal end portion of the second structure are outside of the cavity;
applying a source of suction to the first proximal port of the first structure to cause tissue to be withdrawn from the cavity through the first lumen of the first structure; and
causing make-up gas to flow into the cavity through the second lumen of the second structure while applying the source of suction to the first structure.

24. The method of claim 23, wherein:
positioning the instrument includes positioning the instrument so the second proximal port of the second structure is exposed to ambient atmosphere; and
causing make-up gas to flow into the cavity includes casing the make-up gas to be drawn into the cavity through the second lumen in response to the withdrawal of the tissue through the first lumen.

25. The method of claim 23, wherein the method is performed in a cavity of the subject including a deflated lung of the subject.

26. The method of claim 25, further including operating the instrument in a manner to not cause re-inflation of the lung of the subject.

27. The method of claim 25, further comprising applying a source of gas at a pressure greater than atmospheric pressure to the second proximal port of the second structure.

28. A method for performing a surgical procedure on a subject, comprising:
inserting the instrument of claim 22 into a cavity of the subject, optionally a cavity of the subject's thorax;
positioning the instrument so the distal end portion of the first structure and the distal end portion of the second structure are within the cavity, and the proximal end portion of the first structure and the proximal end portion of the second structure are outside of the cavity;
applying a source of suction to the first proximal port of the first structure to cause tissue to be withdrawn from the cavity through the first lumen of the first structure; and
causing make-up gas to flow into the cavity through the second lumen of the second structure while applying the source of suction to the first structure.

29. A method, comprising:
providing a first structure defining a first lumen and including a first proximal end portion including a first proximal port to the first lumen and a first distal end portion including a first distal port to the first lumen;
providing a second structure defining a second lumen and including a second proximal end portion including a second proximal port to the second lumen and a second distal end portion including a second distal port to the second lumen;
inserting the first structure into a cavity in a subject through an opening in the subject's body, including positioning the first distal port in the cavity;
applying suction to the first proximal port of the first structure to suction tissue from the cavity through the first distal port and the first lumen;
inserting the second structure into the cavity of the subject through an opening in the subject's body, including positioning the second distal port in the cavity, and positioning the second proximal port outside of the cavity; and
wherein make-up gas flows into the cavity through the second lumen and the second distal port while the suction is applied to the first proximal port of the first structure.
